(19) United States
(12) Patent Application Publication
(10) Pub. No.: US 2020/0123555 A1
(43) Pub. Date: Apr. 23, 2020

(54) USE OF MALONYLTRANSFERASE GENE

(71) Applicant: Suntory Holdings Limited, Osaka-shi, Osaka (JP)

(72) Inventors: Naoko OKITSU, Soraku-gun, Kyoto (JP); Noriko NAKAMURA, Soraku-gun, Kyoto (JP)

(73) Assignee: Suntory Holdings Limited, Osaka-shi, Osaka (JP)

(21) Appl. No.: 16/627,840

(22) PCT Filed: Jul. 2, 2018
(86) PCT No.: PCT/JP2018/025101

§ 371 Date: Dec. 31, 2019

(30) Foreign Application Priority Data
Jul. 3, 2017 (JP) 2017-130396

(51) Int. Cl. C12N 15/82; A01H 5/02; A01H 3/00

(52) U.S. Cl. CPC C12N 15/8241; C07D 311/30; A01H 3/00; A01H 5/02

(57) ABSTRACT

An object of the present invention is to provide a method for producing a flavone wherein a malonyl group is added to a glucose at position 7 using a polynucleotide encoding a protein having activity that transfers a malonyl group to position 6 of the glucose at position 7 of flavone 7-glucoside, and create a plant variety having a modified flower color, and preferably a flower color that is bluer than that of existing varieties, by containing as a co-pigment a flavone wherein a malonyl group has been added to the glucose at position 7. A polynucleotide is used that is selected from the group consisting of, for example: (a) a polynucleotide consisting of the base sequence of SEQ ID NO: 1; (b) a polynucleotide that hybridizes under stringent conditions with a polynucleotide consisting of the base sequence complementary to the base sequence of SEQ ID NO: 1 and encodes a protein having activity that transfers a malonyl group to position 6 of the glucose at position 7 of flavone 7-glucoside; (c) a polynucleotide that encodes a protein consisting of the amino acid sequence of SEQ ID NO: 2; and, (d) a polynucleotide encoding a protein that consists of an amino acid sequence wherein one or a few amino acids have been deleted, substituted, inserted and/or added in the amino acid sequence of SEQ ID NO: 2 and has activity that transfers a malonyl group to position 6 of the glucose at position 7 of flavone 7-glucoside.

### FIELD

The present invention relates to a method for creating a plant variety having a modified flower color, and preferably a flower color that is bluer than existing varieties, by containing a flavone having a malonyl group added to glucose at position 7 as a co-pigment using a polynucleotide encoding a protein having activity that transfers a malonyl group to position 6 of the glucose at position 7 of flavone 7-glucoside, and a method for producing a flavone wherein a malonyl group has been added to glucose at position 7.

### BACKGROUND

Flower color is attributable to four types of substances consisting of flavonoids, carotenoids, chlorophyll and betalains. Among these, flavonoids exhibit a diverse range of colors such as yellow, red, violet and blue. Among these flavonoids, group 1 flavonoids exhibit the colors red, violet and blue, are referred to as anthocyanins, and are classified into the three groups of pelargonidin, cyanidin and delphinidin.

In addition to accumulating delphinidin, it is thought that any of (i) modification of anthocyanidin by one or a plurality of aromatic acyl groups, (ii) anthocyanin being present together with a co-pigment such as a flavone or flavonol, (iii) anthocyanin being present together with an iron ion or aluminum ion, (iv) the pH of the vacuole in which the anthocyanin is localized rising from neutral to weakly alkaline, or (v) the anthocyanin, co-pigment and metal ion forming a complex, is required for flower color to be blue (and this type of anthocyanin is referred to as metalloanthocyanin) (NPL 1).

Considerable research has been conducted on the biosynthesis pathway of flavonoids and anthocyanins and associated biosynthetic enzymes and genes encoding these enzymes have been identified (NPL 2). In addition, flavones, which are a type of flavonoid, are known to have the effect of causing a deeper blue color when present together with anthocyanin, and genes encoding biosynthetic enzymes of flavones have been identified from numerous plants.

Enzyme genes that modify anthocyanins and flavones have also been obtained from numerous plants, examples of which include glucosyltransferase genes, acyltransferase genes and methyltransferase genes. Anthocyanins and flavones are subjected to a diverse range of species-specific and variety-specific modifications by these enzymes and this diversity is responsible for the diverse range of flower colors. For example, a gene that encodes a protein having activity that transfers a malonyl group to position 6 of the glucose at position 3 of anthocyanin 3-glucoside has been isolated from chrysanthemum, dahlia and cineraria (NPL 3, PTL 1). A gene that encodes a protein having activity that transfers to position 6 of the glucose at position 5 of anthocyanin 5-glucoside has been isolated from scarlet sage and thale cress (NPL 4, PTL 1). A gene that encodes a protein having activity that transfers a malonyl group to position 6 of the glucose at position 7 of isoflavone 7-glucoside has been isolated from soybean and alfalfa (NPL 5,6). A gene that encodes a protein having activity that transfers a malonyl group to position 6 of the glucose at position 7 of flavonol 7-glucoside has been isolated from tobacco (NPL 7).

### CITATION LIST

### Patent Literature

[PTL 1] International Publication No. WO 2001/092536
[PTL 2] International Publication No. WO 2017/002945

### Non-Patent Literature

[NPL 1] Natural Product Reports (2009), 26 884-915
[NPL 2] Biosci. Biotechnol. Biochem. (2010), 74(9), 1760-1769
[NPL 3] Plant Biotechnology, 20(3), 229-234 (2003)
[NPL 4] The Plant Journal (2007), 50, 678-695
[NPL 5] Phytochemistry 68 (2007), 2035-2042
[NPL 6] The Plant Journal (2008), 55, 382-396
[NPL 7] The Plant Journal (2005), 42, 481-491
[NPL 8] The Journal of Biological Chemistry, Vol. 282, No. 21, 15812-15822

### SUMMARY

### Technical Problem

Blue chrysanthemums that form a derivative of delphinidin blue pigment have been created through genetic recombination (PTL 2). Factors causing this blue flower color are thought to consist of the formation of delphinidin along with the co-pigment effect of luteolin 7-malonylglucoside, a type of flavone, that is present together with the delphinidin. However, a gene derived from chrysanthemum encoding a protein having activity that transfers a malonyl group to position 6 of the glucose at position 7 of luteolin 7-glucoside has yet to be identified.

Under such circumstances, a problem to be solved by the present invention is to identify a polynucleotide that encodes a protein having activity that specifically transfers a malonyl group to position 6 of the glucose at position 7 of flavone 7-glucoside in chrysanthemum, and use this polynucleotide to provide a method for producing a flavone wherein a malonyl group has been added to glucose at position 7. Moreover, an object of the present invention is to form a flavone wherein a malonyl group has been added to the glucose at position 7 using this polynucleotide and create a plant variety having a modified flower color, and preferably a flower color that is bluer than that of existing varieties, due to the co-pigment of this flavone.

### Solution to Problem

As a result of conducting extensive studies and experiments to solve the aforementioned problems, the inventor of the present application identified an anthocyanin malonyltransferase homolog (Dm3MaT3) derived from chrysanthemum acting as a protein having activity that specifically transfers a malonyl group to position 6 of the glucose at position 7 of flavone 7-glucoside, thereby leading to completion of the present invention.

Namely, the present invention is as indicated below.

[1] A method for producing a genetically engineered plant or progeny thereof that produces a flavone wherein a malonyl group is added to glucose at position 7, comprising introducing a polynucleotide selected from the group consisting of the following (a) to (e) into a host plant:

(a) a polynucleotide consisting of the base sequence of SEQ ID NO: 1;

(b) a polynucleotide that hybridizes under stringent conditions with a polynucleotide consisting of the base sequence complementary to the base sequence of SEQ ID NO: 1 and encodes a protein having activity that specifically transfers a malonyl group to position 6 of the glucose at position 7 of flavone 7-glucoside;

(c) a polynucleotide that encodes a protein consisting of the amino acid sequence of SEQ ID NO: 2;

(d) a polynucleotide encoding a protein that consists of an amino acid sequence wherein one or a few amino acids have been deleted, substituted, inserted and/or added in the amino acid sequence of SEQ ID NO: 2 and has activity that specifically transfers a malonyl group to position 6 of the glucose at position 7 of flavone 7-glucoside; and,

(e) a polynucleotide that has an amino acid sequence having identity of 90% or more with respect to the amino acid sequence of SEQ ID NO: 2 and encodes a protein having activity that specifically transfers a malonyl group to position 6 of the glucose at position 7 of flavone 7-glucoside.

[2] The method described in 1, wherein the flavone is luteolin or apigenin.

[3] The method described in 1 or 2, wherein the genetically engineered plant has a modified flower color.

[4] A genetically engineered plant or progeny thereof that produces a flavone wherein a malonyl group is added to glucose at position 7, or a portion or tissue thereof, comprising a polynucleotide selected from the group consisting of the following (a) to (e):

(a) a polynucleotide consisting of the base sequence of SEQ ID NO: 1;

(b) a polynucleotide that hybridizes under stringent conditions with a polynucleotide consisting of the base sequence complementary to the base sequence of SEQ ID NO: 1 and encodes a protein having activity that specifically transfers a malonyl group to position 6 of the glucose at position 7 of flavone 7-glucoside;

(c) a polynucleotide that encodes a protein consisting of the amino acid sequence of SEQ ID NO: 2;

(d) a polynucleotide encoding a protein that consists of an amino acid sequence wherein one or a few amino acids have been deleted, substituted, inserted and/or added in the amino acid sequence of SEQ ID NO: 2 and has activity that specifically transfers a malonyl group to position 6 of the glucose at position 7 of flavone 7-glucoside; and,

(e) a polynucleotide that has an amino acid sequence having identity of 90% or more with respect to the amino acid sequence of SEQ ID NO: 2 and encodes a protein having activity that specifically transfers a malonyl group to position 6 of the glucose at position 7 of flavone 7-glucoside.

[5] The genetically engineered plant or progeny thereof or a part or tissue thereof described in 4, wherein the flavone is luteolin or apigenin.

[6] The genetically engineered plant or progeny thereof or a part or tissue thereof described in 4 or 5, wherein the genetically engineered plant has a modified flower color.

[7] The portion of the plant described in any of 4 to 6 which is a cut flower.

[8] A processed cut flower obtained by using the cut flower described in 7.

[9] A method for producing a flavone wherein a malonyl group is added to glucose at position 7, comprising introducing a polynucleotide selected from the group consisting of the following (a) to (e) into a non-human host:

(a) a polynucleotide consisting of the base sequence of SEQ ID NO: 1;

(b) a polynucleotide that hybridizes under stringent conditions with a polynucleotide consisting of the base sequence complementary to the base sequence of SEQ ID NO: 1 and encodes a protein having activity that specifically transfers a malonyl group to position 6 of the glucose at position 7 of flavone 7-glucoside;

(c) a polynucleotide that encodes a protein consisting of the amino acid sequence of SEQ ID NO: 2;

(d) a polynucleotide encoding a protein that consists of an amino acid sequence wherein one or a few amino acids have been deleted, substituted, inserted and/or added in the amino acid sequence of SEQ ID NO: 2 and has activity that specifically transfers a malonyl group to position 6 of the glucose at position 7 of flavone 7-glucoside; and,

(e) a polynucleotide that has an amino acid sequence having identity of 90% or more with respect to the amino acid sequence of SEQ ID NO: 2 and encodes a protein having activity that specifically transfers a malonyl group to position 6 of the glucose at position 7 of flavone 7-glucoside; and,

culturing or growing the non-human host.

[10] The method described in 9, wherein the non-human host is a plant cell.

[11] The method described in 9 or 10, wherein the flavone is luteolin or apigenin.

### Advantageous Effects of Invention

According to the present invention, a plant variety having a modified flower color, and preferably a flower color that is bluer than that of existing varieties, can be created by containing as co-pigment a flavone wherein a malonyl group has been added to glucose at position 7. In addition, a method for producing a flavone is provided in which a malonyl group has been added to glucose at position 7.

### BRIEF DESCRIPTION OF DRAWINGS

FIG. 1 depicts high-performance liquid chromatograms of reaction solutions obtained by enzymatically reacting each of a protein solution crudely extracted from Escherichia coli expressing Dm3MaT3, a protein solution crudely extracted from Escherichia coli expressing Dm3MaT1 and a protein solution crudely extracted from Escherichia coli expressing Dm3MaT2 with luteolin 7-glucoside.

FIG. 2 depicts high-performance liquid chromatograms of reaction solutions obtained by enzymatically reacting each of a protein solution crudely extracted from Escherichia coli expressing Dm3MaT3, a protein solution crudely extracted from Escherichia coli expressing Dm3MaT1 and a protein solution crudely extracted from Escherichia coli expressing Dm3MaT2 with cyanidin 3-glucoside.

FIG. 3 depicts high-performance liquid chromatograms of reaction solutions obtained by enzymatically reacting a Dm3MaT3 protein solution with luteolin 7-glucoside.

FIG. 4 depicts high-performance liquid chromatograms of reaction solutions obtained by enzymatically reacting a Dm3MaT3 protein solution with luteolin 4′-glucoside.

FIG. 5 is a diagram summarizing the reactivity of Dm3MaT3 protein to various types of flavonoid substrates.

FIG. 6 is an alignment diagram comparing the amino acid sequence of Dm3MaT3 with those of Dm3MaT1 and Dm3MaT2.

FIG. 7 is phylogenetic tree indicating the relationships between the Dm3MaT3 of the present invention and various enzymes.

FIG. 8 is a plasmid map of pSPB7136.

### DESCRIPTION OF EMBODIMENTS

The polynucleotide used in the present invention (SEQ ID NO: 1) encodes Dm3MaT3. In the present description, the term “polynucleotide” refers to DNA or RNA. The polynucleotide used in the present invention is not limited to that consisting of the base sequence of SEQ ID NO: 1 or a polynucleotide encoding a protein comprising the corresponding amino acid sequence thereof (SEQ ID NO: 2), but rather includes a polynucleotide consisting of that base sequence or complementary sequence thereof, or a polynucleotide that encodes a protein having a specific sequence homology, and preferably sequence identity, with that amino acid sequence and has activity that specifically transfers a malonyl group to position 6 of the glucose at position 7 of flavone 7-glucoside.

Although the present description describes a gene that encodes a protein having activity that specifically transfers a malonyl group to position 6 of the glucose at position 7 of flavone 7-glucoside derived from chrysanthemum, the polynucleotide used in the present invention is not limited to a gene derived from chrysanthemum, but rather may have a plant, animal or microorganism as the origin of the gene encoding a protein having activity that specifically transfers a malonyl group to position 6 of the glucose at position 7 of flavone 7-glucoside, and can be used to alter the flower color in a plant regardless of origin provided that the protein has activity that specifically transfers a malonyl group to position 6 of the glucose at position 7 of flavone 7-glucoside.

The polynucleotide used in the present invention includes a polypeptide that hybridizes under stringent conditions with a polynucleotide consisting of a base sequence complementary to the base sequence of SEQ ID NO: 1 and encodes a protein having activity that specifically transfers a malonyl group to position 6 of the glucose at position 7 of flavone 7-glucoside. In the present description, the term “stringent conditions” refers to conditions that enable selective and detectable specific binding between a polynucleotide or oligonucleotide and genome DNA. Stringent conditions are defined by a suitable combination of salt concentration, organic solvent (such as formamide), concentration and other known conditions. Namely, stringency is increased by reducing salt concentration, increasing organic solvent concentration or raising the hybridization temperature. Moreover, washing conditions following hybridization also have an effect on stringency. These washing conditions are also defined according to salt concentration and temperature, and washing stringency increases as a result of reducing salt concentration or raising temperature. Thus, the term “stringent conditions” refers to conditions under which only those base sequences having high homology, such that the degree of identity or homology between each base sequence is in terms of the overall average, for example, approximately 80% or more, preferably approximately 90% or more, more preferably approximately 95% or more, even more preferably approximately 97% or more and most preferably approximately 98% or more, specifically hybridize. An example of “stringent conditions” consists of a temperature of 60° C. to 68° C., sodium concentration of 150 mM to 900 mM and preferably 600 mM to 900 mM, and pH of 6 to 8, and a specific example thereof consists of carrying out hybridization under conditions of 5×SSC (750 mM NaCl, 75 mM trisodium citrate), 1% SDS, 5×Denhardt's solution in 50% formaldehyde and 42° C. followed by carrying out washing under conditions 0.1×SSC (15 mM NaCl, 1.5 mM trisodium citrate), 0.1% SDS and 55° C.

Hybridization can be carried out in accordance with a method known in the art or method complying therewith such as the method described in Current Protocols in Molecular Biology (edited by Frederick M. Ausubelet, et al, 1987. In addition, in the case of using a commercially available library, hybridization can be carried out in accordance with the method described in the user's manual provided therewith. A gene selected as a result of this hybridization may be a gene found in nature such as a plant gene or may be a non-plant gene. In addition, the gene selected as a result of hybridization may be cDNA, genome DNA or chemically synthesized DNA.

The DNA according to the present invention can be obtained by a method known among persons with ordinary skill in the art, such as a method consisting of chemically synthesizing DNA by phosphoramidite or nucleic acid amplification method by using a plant nucleic acid sample as template and using primers designed based on the nucleotide sequence of the target gene.

The polynucleotide used in the present invention includes a polynucleotide encoding a protein that consists of an amino acid sequence wherein one or a few amino acids have been deleted, substituted, inserted and/or added in the amino acid sequence of SEQ ID NO: 2 and has activity that specifically transfers a malonyl group to position 6 of the glucose at position 7 of flavone 7-glucoside. The aforementioned “amino acid sequence wherein one or a few amino acids have been deleted, substituted, inserted and/or added” refers to an amino acid sequence in which an arbitrary number of amino acids such as 1 to 20, preferably 1 to 5 and more preferably 1 to 3 amino acids have been deleted, substituted, inserted and/or added. Site-directed mutagenesis, which is a type of genetic engineering technique, is useful since this technique makes it possible to introduce a specific mutation at a specific site, and can be carried out in compliance with, for example, the method described in Molecular Cloning: A Laboratory Manual, 2nd Ed., Cold Spring Harbor Laboratory Press, Cold Spring Harbor, N.Y., 1989. A protein can be obtained that is consisting of an amino acid sequence wherein one or a few amino acids have been deleted, substituted, inserted and/or added by expressing this mutant DNA using a suitable expression system.

The polypeptide used in the present invention includes a polypeptide encoding a protein that has an amino acid sequence having identity of 90% or more with respect to the amino acid sequence of SEQ ID NO: 2 and has activity that specifically transfers a malonyl group to position 6 of the glucose at position 7 of flavone 7-glucoside. This polypeptide encodes a protein having identity of preferably approximately 95% or more, more preferably approximately 97% or more and most preferably approximately 98% or more with respect to the amino acid sequence of SEQ ID NO: 2. In the present description, the term “identity” refers to the quantity (number) of amino acid residues or bases that can be determined to be identical in the mutual compatibility relationship between each amino acid residue or base that composes the strands between two strands in a polypeptide sequence (or amino acid sequence) or polypeptide sequence (or base sequence), indicates the degree of sequence correlation between two polypeptide sequences or two polynucleotide sequences, and can be easily calculated. There are many known methods for measuring homology between two polynucleotide sequences or two polypeptide sequences, and the term “identity” is widely known among persons with ordinary skill in the art (see, for example, Lesk, A. M. (Ed.), Computational Molecular Biology, Oxford University Press, New York (1988); Smith, D. W. (Ed.), Biocomputing: Informatics and Genome Projects, Academic Press, New York (1993); Griffin, A. M. & Griffin, H. G. (Ed.), Computer Analysis of Sequence Data: Part I, Human Press, New Jersey (1994); von Heinje, G., Sequence Analysis in Molecular Biology, Academic Press, New York (1987); Gribskov, M. & Devereux, J. (Ed.), Sequence Analysis Primer, M-Stockton Press, New York (1991)).

In addition, although the numerical value of “identity” described in the present description is, unless specifically indicated otherwise, the value calculated using a homology search program commonly known among persons with ordinary skill in the art, is it preferably the value calculated using the MacVector application, ClustalW Program (Version 14.5.2(24), Oxford Molecular Ltd., Oxford, England).

The polynucleotide (nucleic acid or gene) used in the present invention “encodes” a protein of interest. Here, “encodes” refers to expressing a protein of interest in a state in which it is provided with the activity thereof. In addition, “encodes” includes both encoding a protein of interest as a contiguous structural sequence (exon) and encoding the protein of interest through an intervening sequence (intron).

Genes having a base sequence found in nature are obtained by analyzing with, for example, a DNA sequencer as described in the subsequent examples. In addition, DNA encoding an enzyme having a modified amino acid sequence can be synthesized using commonly used site-directed mutagenesis or PCR based on DNA having a base sequence found in nature. For example, after obtaining a DNA fragment desired to be modified by subjecting naturally-occurring cDNA or genome DNA to restrictase treatment, the resulting fragment is used as a template to carry out site-directed mutagenesis or PCR using primers introduced with a desired mutation and obtain the desired modified DNA fragment. Subsequently, a DNA fragment that encodes another portion of the enzyme targeting this DNA fragment introduced with a mutation is then linked thereto.

Alternatively, in order to obtain DNA encoding an enzyme consisting of a shortened amino acid sequence, DNA encoding an amino acid sequence that is longer than the target amino acid sequence, such as a full-length amino acid sequence, is cleaved with a desired restrictase, and in the case the resulting DNA fragment does not encode the entire target amino acid sequence, a DNA fragment consisting of the missing sequence is then synthesized and linked thereto.

In addition, the resulting polynucleotide can be confirmed to encode a protein having activity that specifically transfers a malonyl group to position 6 of the glucose at position 7 of flavone 7-glucosidase by expressing the resulting polynucleotide in Escherichia coli or yeast using a gene expression system. Moreover, a protein, which is a product of the polynucleotide, can be obtained that has activity that specifically transfers a malonyl group to position 6 of the glucose at position 7 of flavone 7-glucoside by expressing that polynucleotide. Alternatively, a protein can also be acquired that has activity that specifically transfers a malonyl group to position 6 of the glucose at position 7 of flavone 7-glucoside by using an antibody to a polypeptide consisting of the amino acid sequence described in SEQ ID NO: 2, and a polynucleotide can be cloned that encodes a protein having activity that specifically transfers a malonyl group to position 6 of the glucose at position 7 of flavone 7-glucoside derived from another organism using that antibody.

A flavone wherein a malonyl group has been added to the glucose at position 7 can be contained as a co-pigment by introducing an exogenous polynucleotide encoding a protein having activity that specifically transfers a malonyl group to position 6 of the glucose at position 7 of flavone 7-glucoside obtained in this manner into, for example, a (recombinant) vector, and particularly an expression vector, thereby enabling the production of a genetically engineered plant, progeny thereof, portion thereof or tissue thereof (including cells) that has a modified flower color, and preferably a flower color that is bluer than that of existing varieties. The form of the portion or tissue can be a cut flower.

Examples of transformable plants include, but are not limited to, rose, carnation, chrysanthemum, snapdragon, cyclamen, orchid, eustoma, freesia, gerbera, gladiola, baby's breath, kalanchoe, lily, pelargonium, geranium, petunia, torenia, tulip, anthurium, moth orchid, rice, barley, wheat, rape, potato, tomato, poplar, banana, eucalyptus, sweet potato, soybean, alfalfa, lupine, corn, cauliflower and dahlia.

In addition, according to the present invention, a processed product (processed cut flower) is provided by using a cut flower of the genetically engineered plant or progeny thereof obtained in accordance with that described above. Here, a processed cut flower includes, but is not limited to, a pressed flower, preserved flower, dry flower or resin-sealed flower obtained using that cut flower.

Moreover, a flavone wherein a malonyl group has been added to the glucose at position 7 can be easily produced by introducing a polynucleotide encoding a protein having the ability to specifically transfer a malonyl group to position 6 of the glucose at position 7 of flavone 7-glucoside into a non-human host containing a malonyl group donor such as flavone 7-glucoside and/or malonyl CoA, culturing or growing the non-human host, and harvesting the flavone in which a malonyl group has been added to the glucose at position 7 from the non-human host.

A target protein can be obtained by recovering and purifying from a culture or medium obtained by culturing, cultivating or growing the transformed non-human host in accordance with routine methods, such as filtration, centrifugal separation, cell disruption, gel filtration chromatography or ion exchange chromatography. In addition, the flavone in which a malonyl group has been added to the glucose at position 7 produced according to the production method of the present invention can be used in the production of foods, pharmaceuticals or cosmetics and the like.

A prokaryote or eukaryote can be used as a non-human host. Examples of prokaryotes that can be used include routinely used bacterial hosts such as bacteria belonging to the genus Escherichia such as Escherichia coli and microorganisms of the genus Bacillus such as Bacillus subtilis. Examples of eukaryotes that can be used lower eukaryotes such as eukaryotic microorganisms in the manner of, for example, fungi such as yeast or filamentous fungi.

Examples of yeast include Saccharomyces cerevisiae, while examples of filamentous fungi include microorganisms of the genus Aspergillus such as Aspergillus oryzae or Aspergillus niger and microorganisms of the genus Penicillium. Animal cells or plant cells can also be used for the host, mouse, hamster, monkey or human cells are used as animal cells, and insect cells such as silkworm cells or adult silkworms per se are also used as hosts. Plant cells are preferably used in the method of the present invention.

At the present level of technology, technology can be used to constitutively or tissue-specifically express the aforementioned polynucleotide by introducing the polynucleotide into a non-human host using a (recombinant) vector, and particularly an expression vector, comprising that polynucleotide. Introduction of the polynucleotide into a non-human host can be carried out by a method known among persons with ordinary skill in the art such as the Agrobacterium method, binary vector method, electroporation method, PEG method or particle gun method.

The expression vector used in the present invention contains expression control regions, such as a promoter, terminator and replication origin, that are dependent on the type of non-human host into which the vector is introduced. A routinely used promoter such as a trc promoter, tac promoter or lac promoter is used for the promoter of bacterial expression vectors, while a promoter such as glyceryl aldehyde 3 phosphate dehydrogenase promoter or PHO5 promoter is used as a yeast promoter, and a promoter such as amylase promoter or trpC promoter is used as a promoter for filamentous fungi. In addition, a promoter such as SV40 early promoter or SV40 late promoter is used as a viral promoter.

Examples of promoters that constitutively express polynucleotides in plant cells include cauliflower mosaic virus 35S RNA promoter, rd29A gene promoter, rbcS promoter and mac-1 promoter. In addition, a promoter of a gene that is specifically expressed in a tissue can be used to express a tissue-specific gene.

Expression vectors can be produced by carrying out in accordance with routine methods using enzymes such as restrictases and ligases.

### EXAMPLES

The following provides a detailed explanation of the present invention in accordance with examples.

### Example 1: Experiment Measuring Enzyme Activity of Protein Candidates Having Activity that Transfers a Malonyl Group to Position 6 of the Glucose at Position 7 of Flavone 7-Glucoside (Case of Using Protein Solutions Crudely Extracted from Escherichia coli)

<Production of Escherichia coli Expression Vector>

An Escherichia coli expression vector (pET32a-DM3MaT3) containing the full length of Dm3MaT3 was produced in accordance with the protocol recommended by the manufacturer using Dm3MaT3, for which function has yet to be identified as described in NPL 8, as a protein candidate having activity that transfers a malonyl group to position 6 of the glucose at position 7 of flavone 7-glucoside and using pET32a (Novagen).

<Expression of Malonyltransferase in Escherichia coli>

pET32a-Dm3aT3 was introduced in Escherichia coli strain BL21 using One Shot BL21 (DE3) (Invitrogen) in accordance with the protocol recommended by the manufacturer to acquire transformed Escherichia coli. This Escherichia coli was cultured using the Overnight Expression Autoinduction System 1 (Novagen) in accordance with the protocol recommended by the manufacturer. The transformed Escherichia coli was cultured at 37° C. in 2 ml of prepared culture broth until the OD600 value reached 0.5 (approximately 4 hours). This Escherichia coli broth was used as pre-culture broth and added to 50 ml of culture broth followed by culturing overnight at 25° C.

After culturing overnight, the Escherichia coli broth was centrifuged (3000 rpm, 4° C., 15 minutes) and the harvested bacterial cells were suspended in 5 ml of sonic buffer (composition: 2.5 mM Tris HCl (pH 7.0), 1 mM dithiothreitol (DTT), 10 μM amidinophenylmethanesulfonyl fluoride hydrochloride (APMSF)) followed by disrupting the Escherichia coli by ultrasonication, centrifuging (15000 rpm, 4° C., 10 minutes) and recovering the supernatant. The recovered supernatant was a protein solution crudely extracted from Escherichia coli expressing Dm3MaT3. The Avanti HP-26XP (rotor: JA-2) (Beckman Coulter) was used for centrifugation.

<Measurement of Enzyme Activity>

A reaction solution, obtained by mixing 50 μl of a protein solution crudely extracted from Escherichia coli expressing Dm3MaT3, 5 μl of 1 mg/ml malonyl-CoA, 5 μl of 1 M KPB (pH 7.0), 5 μl of 500 μg/ml luteolin 7-glucoside (dissolved in a 50% aqueous acetonitrile solution containing 0.1% TFA) and adjusting to a volume of 100 μl with water on ice, was held for 20 minutes at 30° C. Subsequently, 100 μl of stop buffer (90% aqueous acetonitrile solution containing 0.1% TFA) were added to stop the reaction followed by analyzing the reaction solution by high-performance liquid chromatography (Prominence, Shimadzu Corp.). Flavone was detected at 330 nm using the Shimadzu PDA SPD-M20A for the detector. The Shim-Pack ODS 150 mm×4.6 mm column (Shimadzu Corp.) was used for the column. Solution A (0.1% aqueous TFA solution) and Solution B (90% aqueous methanol solution containing 0.1% TFA) were used for elution. Elution was carried out by eluting over a linear concentration gradient from an 8:2 mixture to a 0:10 mixture of both solutions for 16 minutes followed by eluting with a 0:10 mixture for 6 minutes. The flow rate was 0.6 ml/min. A protein solution crudely extracted from Escherichia coli introduced with pET32a vector not inserted with an insert was used to carry out the experiment in the same way for use as a control.

Chrysanthemum has activity that transfers a malonyl group to position 6 of the glucose at position 3 of anthocyanin in addition to activity that transfers a malonyl group to position 6 of the glucose at position 7 of flavone 7-glucosidase (see NPL 8). In order to distinguish Dm3MaT3 from chrysanthemum-derived anthocyanin 3-O-glucoside-6″-O-malonyltransferase (Dm3MaT1) and chrysanthemum-derived anthocyanin 3-O-glucoside-3″,6″-O-dimalonyltransferase (Dm3MaT2), which have previously been reported as a gene that encodes a protein having activity that transfers a malonyl group to position 6 of the glucose at position 3 of anthocyanin and a gene that encodes a protein having activity that transfers a malonyl group to position 3 of the glucose at position 3 of anthocyanin in which position 6 of the glucose at position 3 has been malonylated, Escherichia coli expression vectors were similarly prepared for Dm3MaT1 and Dm3MaT2 and experiments for measuring enzyme activity were carried out using protein solutions crudely extracted from Escherichia coli. Moreover, an enzyme reaction using cyanidin 3-glucoside as substrate was also carried out and compared with the result for Dm3MaT3. In that case, when analyzing the reaction solution by high-performance liquid chromatography (Prominence, Shimadzu Corp.), the Shimadzu PDA SPD-M20A was used for the detector and anthocyanin was detected at 520 nm. The Shodex RSpak DE-413L column (Shodex) was used for the column. Solution A (0.1% aqueous TFA solution) and Solution B (90% aqueous acetonitrile solution containing 0.1% TFA) were used for elution. Elution was carried out by eluting over a linear concentration gradient from an 8:2 mixture to a 0:10 mixture of both solutions for 15 minutes followed by eluting with a 0:10 mixture for 5 minutes. The flow rate was 0.6 ml/min.

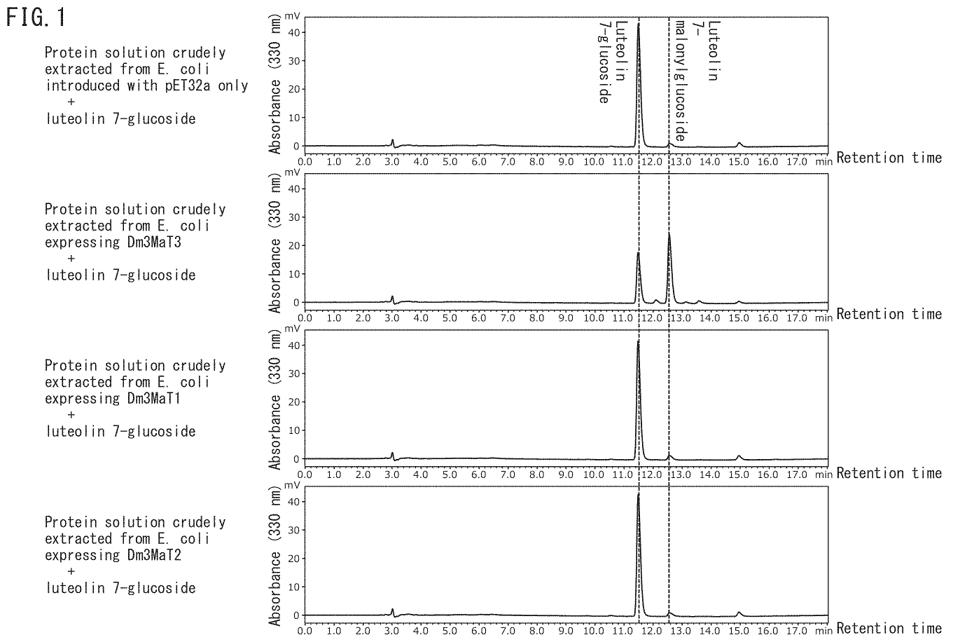

As a result, a peak corresponding to luteolin 7-malonylglucoside was detected in addition to luteolin 7-glucoside added as substrate when Dm3MaT3 was enzymatically reacted with luteolin 7-glucoside. A peak corresponding to luteolin 7-malonylglucoside was not detected in the case of having enzymatically reacted luteolin 7-glucoside with Dm3MaT1 or Dm3MaT2 (see FIG. 1).

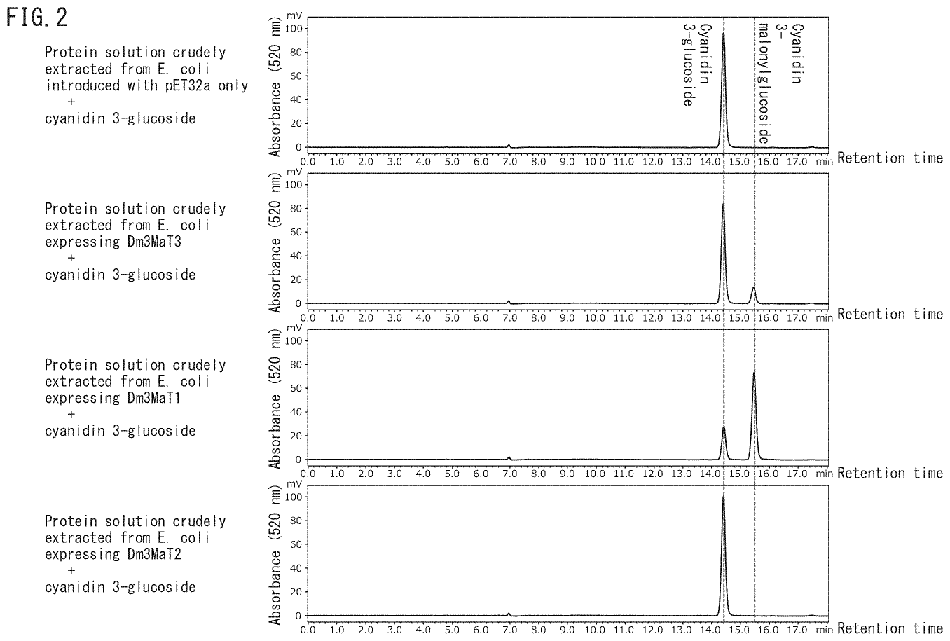

In addition, a peak corresponding to cyanidin 3-malonylglucoside was detected in addition to cyanidin 3-glucoside added as substrate when Dm3MaT3 was enzymatically reacted with cyanidin 3-glucoside. However, in comparison with the case of having reacted Dm3MaT1 with cyanidin 3-glucoside, the amount of cyanidin 3-glucoside consumed was clearly less than in the case of Dm3MaT3 (see FIG. 2).

On the basis of these results, Dm3MaT3 differs from Dm3MaT1 and Dm3MaT2 in that the possibility was indicated that Dm3MaT3 is a gene that encodes a protein having activity that transfers a malonyl group to position 6 of the glucose at position 7 of flavone 7-glucoside and not a gene that encodes a protein having activity that transfers a malonyl group to position 6 of the glucose at position 3 of anthocyanin 3-glucoside or a gene that encodes a protein having activity that transfers a malonyl group to position 3 of the glucose at position 3 of anthocyanin in which position 6 of the glucose at position 3 has been malonylated.

### Experiment 2: Experiment Measuring Enzyme Activity of Proteins Having Activity that Transfers a Malonyl Group to Position 6 of the Glucose at Position 7 of Flavone 7-Glucoside (Case of Using Protein Solution Obtained by Purifying His-Tagged Protein from Escherichia coli)

<Expression of Malonyltransferase in Escherichia coli and Protein Purification>

The Escherichia coli strain BL21 introduced with pET32a-Dm3MaT3 described in Example 1 was cultured using the Overnight Express Autoinduction System 1 (Novagen) in accordance with the protocol recommended by the manufacturer. The transformed Escherichia coli was cultured at 37° C. in 8 ml of prepared culture broth until the OD600 value reached 0.5 (approximately 4 hours). This Escherichia coli broth was used as pre-culture broth and added to 200 ml of culture broth followed by culturing overnight at 25° C.

After culturing overnight, the Escherichia coli broth was centrifuged (1000×g, 4° C., 10 minutes) and the harvested bacterial cells were suspended in 20 ml of extraction buffer (composition: 300 mM KCl, 50 mM KH₂PO₄, 5 mM imidazole (pH 8.0), 10 μM amidinophenylmethanesulfonyl fluoride hydrochloride (APMSF)) followed by disrupting the Escherichia coli by ultrasonication, centrifuging (1400×g, 4° C., 20 minutes) and recovering the supernatant. The supernatant was passed through a 0.45 μm and subjected to His-Tagged purification using Profinia (Bio-Rad) in accordance with the protocol recommended by the manufacturer. The resulting purified protein solution was centrifuged (7500×g, 4° C., 15 minutes) using Centrifugal Filters (Ultracel-10K, Amicon Ultra) and the concentrated protein solution was used as “Dm3MaT3 protein solution”. The Avanti HP-26XP (rotor: JA-2) (Beckman Coulter) was used for centrifugation.

<Measurement of Enzyme Activity>

A reaction solution, obtained by mixing 30 μl of Dm3MaT3 protein solution (10 μg), 5 μl of 1 mg/ml malonyl-CoA, 5 μl of 1 M KPB (pH 7.0), 5 μl of 500 μg/ml luteolin 7-glucoside (dissolved in a 50% aqueous acetonitrile solution containing 0.1% TFA) and adjusting to a volume of 100 μl with water on ice, was held for 20 minutes at 30° C. Subsequently, 100 μl of stop buffer (90% aqueous acetonitrile solution containing 0.1% TFA) were added to stop the reaction followed by analyzing the reaction solution by high-performance liquid chromatography (Prominence, Shimadzu Corp.). Flavone was detected at 330 nm using the Shimadzu PDA SPD-M20A for the detector. The Shim-Pack ODS 150 mm×4.6 mm column (Shimadzu Corp.) was used for the column. Solution A (0.1% aqueous TFA solution) and Solution B (90% aqueous methanol solution containing 0.1% TFA) were used for elution. Elution was carried out by eluting over a linear concentration gradient from an 8:2 mixture to a 0:10 mixture of both solutions for 16 minutes followed by eluting with a 0:10 mixture for 6 minutes. The flow rate was 0.6 ml/min.

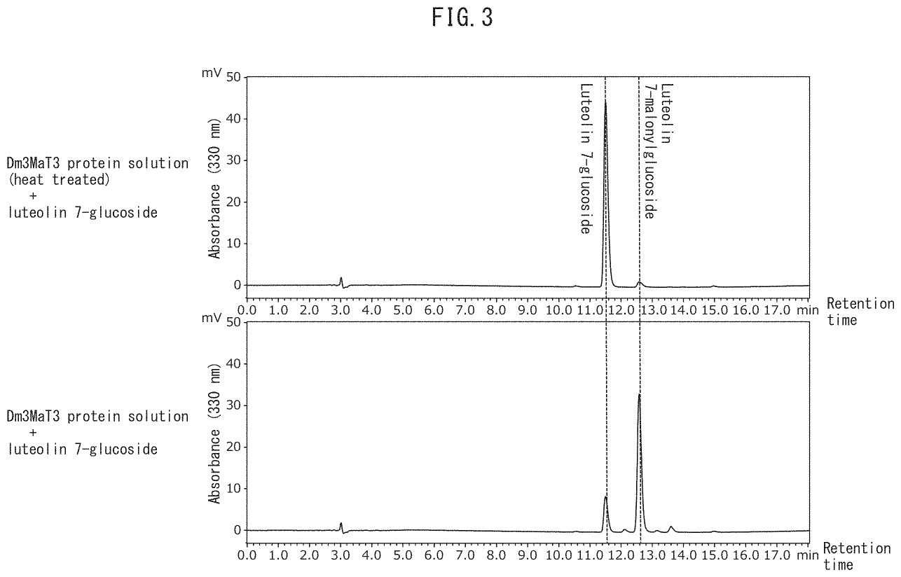

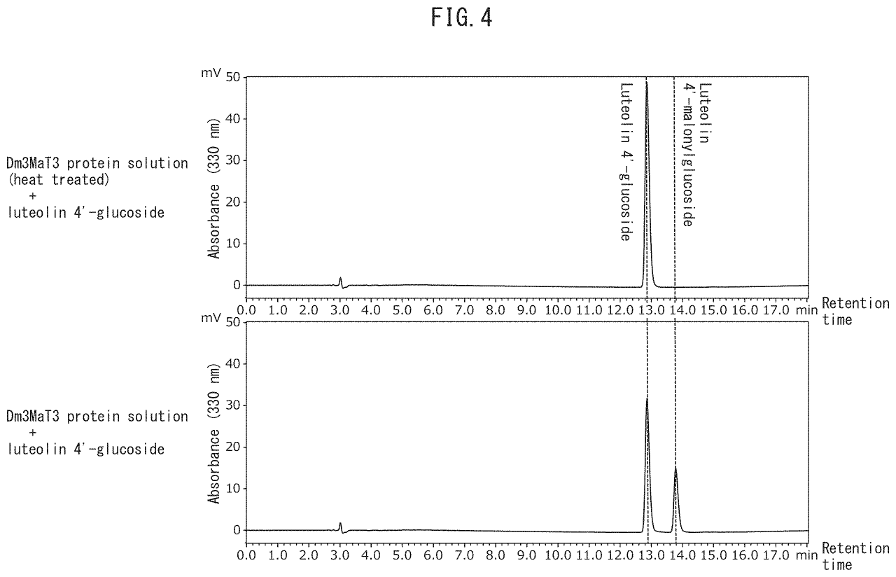

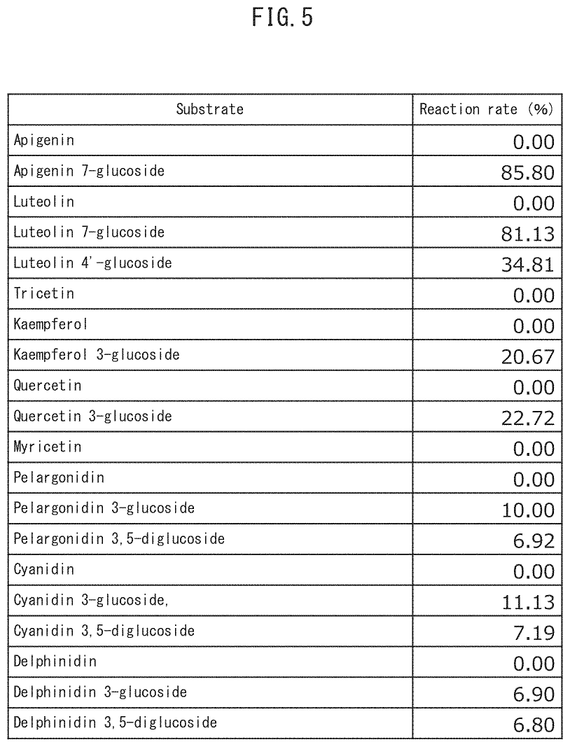

As a result, luteolin 7-malonylglucoside was synthesized in the enzyme reaction solution. The reaction rate (percentage of substrate converted) was 81.13% (see FIGS. 3 and 5). Apigenin 7-malonylglucoside was synthesized in the case of having carried out an enzymatic reaction under the same reaction conditions using 500 μg/ml of apigenin 7-glucoside (dissolved in a 50% aqueous acetonitrile solution containing 0.1% TFA) for the substrate. The reaction rate was 85.80% (FIG. 5). On the other hand, although a substance predicted to be luteolin 4′-malonylglucosde was synthesized in the case of having carried out an enzyme reaction under the same reaction conditions using 500 μg/ml of luteolin 4′-glucoside (dissolved in a 50% aqueous acetonitrile solution containing 0.1% TFA) for the substrate, the reaction rate was only 34.81% (see FIGS. 4 and 5). Moreover, when reactivity was investigated for the various types of flavonoid compounds listed in FIG. 5 (apigenin, luteolin, tricetin, kaempferol, kaempferol 3-glucoside, quercetin, quercetin 3-glucoside, myricetin, pelargonidin, pelargonidin 3-glucoside, pelargonidin 3,5-diglucoside, cyanidin, cyanidin 3-glucoside, cyanidin 3,5-diglucoside, delphinidin, delphinidin 3-glucoside and delphinidin 3,5-diglucoside), Dm3MaT3 protein selectively malonylated the glucose at position 7 of flavone 7-glucoside in the manner of apigenin 7-glucoside and luteolin 7-glucoside, and was clearly determined to be a malonyltransferase having high substrate specificity (see FIG. 5).

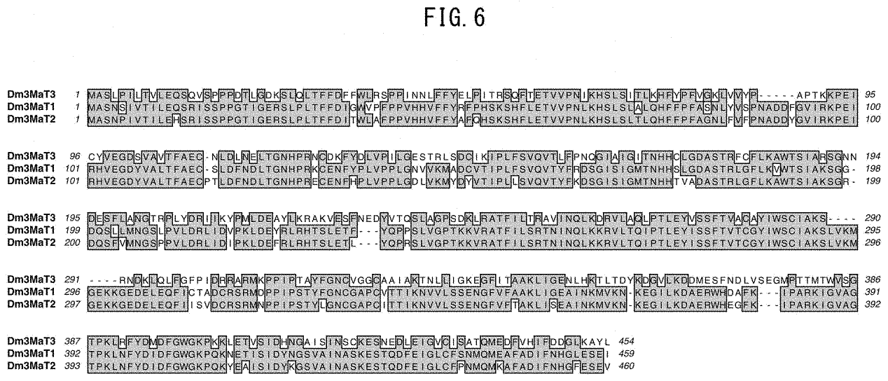

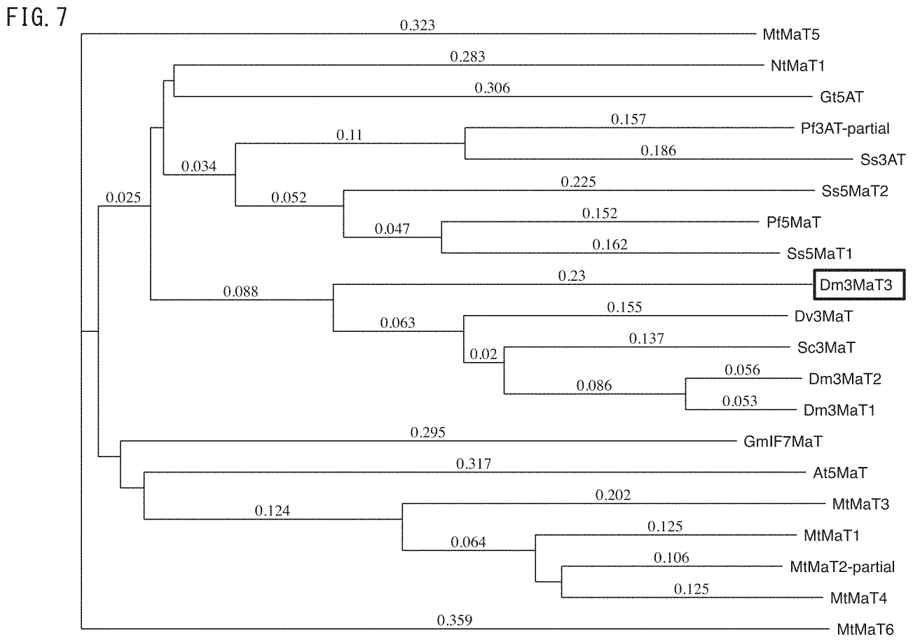

In addition, base sequence and amino acid sequence identity were analyzed between Dm3MaT3 and known glycosyltransferases. When Dm3MaT3 was compared with malonyltransferases derived from the same chrysanthemum, amino acid sequence identity between Dm3MaT3 and Dm3MaT1 and between Dm3MaT3 and Dm3MaT2 were 55% and 53%, respectively (see FIG. 6). The amino acid sequence that exhibits the highest identity with Dm3MaT3 among previously identified malonyltransferases is Dm3MaT1 (see FIG. 7). The MacVector application, ClustalW Program (Version 14.5.2(24), Oxford Molecular Ltd., Oxford, England), was used for this analysis. However, Dm3MaT1 and Dm3MaT2 do not have activity that transfers a malonyl group to position 6 of the glucose at position 7 of flavone 7-glucoside, and the Dm3MaT3 of the present application is a malonyltransferase for which the function thereof differs from that of Dm3MaT1 and Dm3MaT2.

### Experiment 3: Expression of Dm3MaT3 Gene in Rose

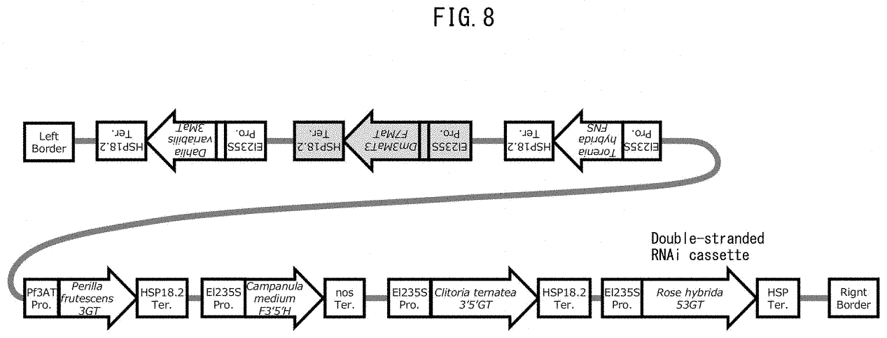

In order to confirm that the Dm3MaT3 gene of the present invention encodes a protein having activity that transfers a malonyl group to position 6 of the glucose at position 7 of flavone 7-glucoside in plants, a binary vector pSPB7136 was constructed to express Dm3MaT3 in plants (see FIG. 8) and subsequently introduced into a rose (variety: Ocean Song). pBINPLUS (Van Engel et al., Transgenic Research 4, 288) was used for the basic skeleton, E1235S promoter (Mitsuhara et al., (1996) Plant Cell Physiol., 37, p. 49) was used for the promoter expressing Dm3MaT3 gene, and HSP terminator (Plant Cell Physiol. (2010) 51, 328-332) was used for the terminator in pSPB7136.

Expression of Dm3MaT3 gene was analyzed using the young leaves of a genetically engineered rose introduced with pSPB7136. Isolation of total RNA was acquired using the Plant RNAeasy Kit (Qiagen) in accordance with the protocol recommended by the manufacturer and cDNA synthesis was carried out using the GeneRacer Kit (Invitrogen) in accordance with the protocol recommended by the manufacturer. Using the cDNA as a template, reverse transcription PCR was carried out with 20 μl using AmpliTaq Gold DNA Polymerase (Thermo Fisher Scientific) in accordance with the protocol recommended by the manufacturer (by repeating 30 cycles of holding for 5 minutes at 94° C. followed by holding for 30 seconds at 94° C., 30 seconds at 55° C. and 1 minute 30 seconds at 72° C. followed by holding for 7 minutes at 72° C. and finally at 4° C.). At that time, primers (forward primer: ATGGCTTTCTTCCCATCTTG, reverse primer: TTAAAGGTATGCTTTTAGTCC) were designed and used so as to specifically amplify the full-length cDNA of Dm3MaT3. When the reaction product was analyzed by agarose gel electrophoresis, a 1365 bp band corresponding to the full length cDNA was detected, thereby confirming that Dm3MaT3 gene was transcribed in the rose.

### Experiment 4: Functional Analysis of Dm3MaT3 in Rose

A crude enzyme solution was prepared from the petals of a rose strain in which was synthesized the transcription product of full-length cDNA Dm3MaT3 followed by an evaluation of the presence or absence of activity transferring a malonyl group to position 6 of the glucose at position 7 of flavone 7-glucoside. 2.5 g of a flower petal sample were crushed in a mortar while cooling with liquid nitrogen followed by dissolving in 30 ml of extraction buffer (composition: 100 mM Tris HCl (pH 7.5), 10 mg/ml polyvinylpyrrolidone K-30, 1 mg/ml 1-thioglycerol, 10 μM amidinophenylmethanesulfonyl fluoride hydrochloride (APMSF)). The resulting protein solution was centrifuged (10,000 rpm, 4° C., 10 minutes) and ammonium sulfate was added to the recovered supernatant to 35% of the saturated concentration. After stirring for 1 hour at 4° C., the solution was centrifuged (10,000 rpm, 4° C., 10 minutes) and the supernatant was recovered. Ammonium sulfate was added to the resulting supernatant to 70% of the saturated concentration followed by stirring for 3 hours at 4° C. and centrifuging (10,000 rpm, 4° C., 10 minutes) to obtain a precipitate. This precipitate was dissolved in 1 ml of elution buffer (composition: 20 mM Tris HCl (pH 7.5), 1 mM DTT, 10 μM amidinophenylmethanesulfonyl fluoride hydrochloride (APMSF)) followed by column purification using the NAP-5 Column Sephadex G-25 DNA Grade (Ge Healthcare) to remove the ammonium sulfate. This solution was designated as “flower petal crude enzyme solution”. The Avanti HP-26XP (rotor: JA-2) (Beckman Coulter) was used for centrifugation.

A reaction solution, obtained by mixing 20 μl of the flower petal crude enzyme solution, 5 μl of 1 mg/ml malonyl-CoA, 5 μl of 1 M KPB (pH 7.0) and 2.5 μl of 1 mM apigenin (dissolved in 50% aqueous acetonitrile solution containing 0.1% TFA) and adjusting to a volume of 100 μl with water on ice, was held for 20 minutes at 30° C. Subsequently, 100 μl of stop buffer (90% aqueous acetonitrile solution containing 0.1% TFA) were added to stop the reaction followed by analysis of the reaction solution by high-performance liquid chromatography (Prominence, Shimadzu Corp.). Flavone was detected at 330 nm using the Shimadzu PDA SPD-M20A for the detector. The Shim-Pack ODS 150 mm×4.6 mm column (Shimadzu Corp.) was used for the column. Solution A (0.1% aqueous TFA solution) and Solution B (90% aqueous methanol solution containing 0.1% TFA) were used for elution. Elution was carried out by eluting over a linear concentration gradient from an 9:1 mixture to a 8:2 mixture of both solutions for 20 minutes, over a linear concentration gradient from an 8:2 mixture to a 2:8 mixture for 15 minutes, and over a linear concentration gradient from a 2:8 mixture to a 0:10 mixture for 5 minutes followed by eluting with a 0:10 mixture for 1 minute. The flow rate was 0.6 ml/min. A crude enzyme solution was prepared from flower petals in the same manner for a non-genetically engineered rose followed by measurement of enzyme activity for use as a control.

Apigenin accounted for 71.24% and apigenin 7-glucoside accounted for 28.76% of the apigenin compounds in the enzyme reaction solution obtained using the flower petal crude enzyme solution from the non-genetically engineered rose, and apigenin 7-malonylglucoside was not detected. A peak corresponding to 7-malonylglucoside accounted for 2.04% while the remaining 97.96% consisted of apigenin and apigenin 7-glucosode, which were also contained in the enzyme reaction solution obtained using the flower petal crude enzyme solution from the non-genetically engineered rose, in the enzyme reaction solution obtained using the flower petal crude enzyme solution from the genetically engineered rose introduced with Dm3MaT3 gene. On the basis thereof, Dm3MaT3, which has activity that transfers a malonyl group to the glucose at position 7 of flavone 7-glucoside, was confirmed to be expressed in flower petals of the genetically engineered rose.

### SEQUENCE LISTING

1. A method for producing a genetically engineered plant or progeny thereof that produces a flavone wherein a malonyl group is added to glucose at position 7, comprising introducing a polynucleotide selected from the group consisting of the following (a) to (e) into a host plant:
(a) a polynucleotide consisting of the base sequence of SEQ ID NO: 1;
(b) a polynucleotide that hybridizes under stringent conditions with a polynucleotide consisting of the base sequence complementary to the base sequence of SEQ ID NO: 1 and encodes a protein having activity that specifically transfers a malonyl group to position 6 of the glucose at position 7 of flavone 7-glucoside;
(c) a polynucleotide that encodes a protein consisting of the amino acid sequence of SEQ ID NO: 2;
(d) a polynucleotide encoding a protein that consists of an amino acid sequence wherein one or a few amino acids have been deleted, substituted, inserted and/or added in the amino acid sequence of SEQ ID NO: 2 and has activity that specifically transfers a malonyl group to position 6 of the glucose at position 7 of flavone 7-glucoside; and,
(e) a polynucleotide encoding a protein that has an amino acid sequence having identity of 90% or more with respect to the amino acid sequence of SEQ ID NO: 2 and has activity that specifically transfers a malonyl group to position 6 of the glucose at position 7 of flavone 7-glucoside.

2. The method according to claim 1, wherein the flavone is luteolin or apigenin.

3. The method according to claim 1, wherein the genetically engineered plant has a modified flower color.

4. A genetically engineered plant or progeny thereof that produces a flavone wherein a malonyl group is added to glucose at position 7, or a portion or tissue thereof, comprising a polynucleotide selected from the group consisting of the following (a) to (e):
(a) a polynucleotide consisting of the base sequence of SEQ ID NO: 1;
(b) a polynucleotide that hybridizes under stringent conditions with a polynucleotide consisting of the base sequence complementary to the base sequence of SEQ ID NO: 1 and encodes a protein having activity that specifically transfers a malonyl group to position 6 of the glucose at position 7 of flavone 7-glucoside;
(c) a polynucleotide that encodes a protein consisting of the amino acid sequence of SEQ ID NO: 2;
(d) a polynucleotide encoding a protein that consists of an amino acid sequence wherein one or a few amino acids have been deleted, substituted, inserted and/or added in the amino acid sequence of SEQ ID NO: 2 and has activity that specifically transfers a malonyl group to position 6 of the glucose at position 7 of flavone 7-glucoside; and,
(e) a polynucleotide encoding a protein that has an amino acid sequence having identity of 90% or more with respect to the amino acid sequence of SEQ ID NO: 2 and has activity that specifically transfers a malonyl group to position 6 of the glucose at position 7 of flavone 7-glucoside.

5. The genetically engineered plant or progeny thereof, or a part or tissue thereof according to claim 4, wherein the flavone is luteolin or apigenin.

6. The genetically engineered plant or progeny thereof or a part or tissue thereof according to claim 4, wherein the genetically engineered plant has a modified flower color.

7. The portion of the plant according to claim 4 which is a cut flower.

8. A processed cut flower obtained by using the cut flower according to claim 7.

9. A method for producing a flavone wherein a malonyl group is added to glucose at position 7, comprising:
introducing a polynucleotide selected from the group consisting of the following (a) to (e) into a non-human host:
(a) a polynucleotide consisting of the base sequence of SEQ ID NO: 1;
(b) a polynucleotide that hybridizes under stringent conditions with a polynucleotide consisting of the base sequence complementary to the base sequence of SEQ ID NO: 1 and encodes a protein having activity that specifically transfers a malonyl group to position 6 of the glucose at position 7 of flavone 7-glucoside;
(c) a polynucleotide that encodes a protein consisting of the amino acid sequence of SEQ ID NO: 2;
(d) a polynucleotide encoding a protein that consists of an amino acid sequence wherein one or a few amino acids have been deleted, substituted, inserted and/or added in the amino acid sequence of SEQ ID NO: 2 and has activity that specifically transfers a malonyl group to position 6 of the glucose at position 7 of flavone 7-glucoside; and,
(e) a polynucleotide encoding a protein that has an amino acid sequence having identity of 90% or more with respect to the amino acid sequence of SEQ ID NO: 2 and has activity that specifically transfers a malonyl group to position 6 of the glucose at position 7 of flavone 7-glucoside; and,
culturing or growing the non-human host.

10. The method according to claim 9, wherein the non-human host is a plant cell.

11. The method according to claim 9, wherein the flavone is luteolin or apigenin.


SEQUENCE LISTING (8 sequences)

SEQ ID NO: 1 - DNA, 1365 bp, Chrysanthemum x morifolium
atggcttctcttcccatcttgactgttcttgagcagtcccaagtatcgccaccaccagacactctaggcgacaagtcactgcaactcacttttttcgatttcttttggctgcgttctcctccgattaacaatcttttcttttacgagcttcccatcactagaagccaatttactgaaaccgttgttcccaatattaaacattcgttatcgattacacttaaacatttttacccttttgtaggtaagttggtagtatatcctgctcctactaaaaagcctgaaatttgttatgttgaaggtgattctgtggctgttactttcgcagaatgtaaccttgacttaaacgaattaacaggaaaccatcctagaaactgtgacaagttttacgatcttgtaccaatactaggtgagagtactagattatcagactgcataaagattccacttttctccgttcaagtgacgctttttcccaaccaaggaatagctattggaattacaaatcaccattgcctaggtgatgctagcactcggttttgtttcttgaaggcgtggacatcaattgcgcgatctggtaataatgatgagtcatttttagctaacggcactaggccattatatgatagaatcatcaaatatccaatgctagatgaagcgtatctaaagcgtgcaaaagttgaaagttttaatgaagattatgtaactcaaagcctagctggaccgagtgataaacttcgtgccacgtttatcttgacacgagctgttatcaatcagttaaaagatagagtattagcccaactgccaacattagaatatgtatcatcttttacagtagcatgtgcttacatttggagttgcattgcgaaatcacgcaatgataagttgcaactatttggtttcccaattgatcgtagagcacgtatgaagccacctatacccacggcctactttggcaactgcgtcgggggttgtgcggcgattgcaaaaaccaatcttttaatcggaaaagaaggattcataactgctgcaaaattgataggagagaatctacacaagaccttgactgattataaggatggagtcctaaaagacgacatggaatctttcaatgatttggtctctgaaggaatgccaacaacaatgacatgggtatccggaacaccaaagcttagattctatgacatggatttcgggtgggggaagccaaaaaagctcgaaacagtttcaattgatcataatggagcaatatctatcaattcttgcaaagaatcaaatgaagatctggagattggtgtttgcatttcagctactcagatggaggattttgttcatatctttgatgatggactaaaagcatacctttaa

SEQ ID NO: 2 - protein, 454 aa, Chrysanthemum x morifolium
MASLPILTVLEQSQVSPPPDTLGDKSLQLTFFDFFWLRSPPINNLFFYELPITRSQFTETVVPNIKHSLSITLKHFYPFVGKLVVYPAPTKKPEICYVEGDSVAVTFAECNLDLNELTGNHPRNCDKFYDLVPILGESTRLSDCIKIPLFSVQVTLFPNQGIAIGITNHHCLGDASTRFCFLKAWTSIARSGNNDESFLANGTRPLYDRIIKYPMLDEAYLKRAKVESFNEDYVTQSLAGPSDKLRATFILTRAVINQLKDRVLAQLPTLEYVSSFTVACAYIWSCIAKSRNDKLQLFGFPIDRRARMKPPIPTAYFGNCVGGCAAIAKTNLLIGKEGFITAAKLIGENLHKTLTDYKDGVLKDDMESFNDLVSEGMPTTMTWVSGTPKLRFYDMDFGWGKPKKLETVSIDHNGAISINSCKESNEDLEIGVCISATQMEDFVHIFDDGLKAYL

SEQ ID NO: 3 - DNA, 1380 bp, Chrysanthemum x morifolium
atggcttccaattccattgttacaattcttgaacaatctcgaatatcctcaccaccaggcaccattggtgaacgttcattgccacttacttttttcgacattggatgggtacctttccctccagtccaccatgttttcttctaccgcttcccgcactctaaatcacatttcttagaaaccgttgtcccaaatcttaaacactctttatcactcgcactacaacattttttcccattcgctagcaatttgtatgtatctcctaatgctgatgattttggcgtcattagaaaaccggaaattagacacgtggaaggtgattatgttgcacttacttttgcagaatgttctcttgattttaatgatttgacaggaaatcatcctcgaaaatgtgaaaacttttatccacttgtgcctccattaggtaatgttgttaaaatggctgattgtgtcacaatcccacttttttcagtccaagtgacgtactttagggactcgggtatctctattggaatgacaaatcatcatagcctaggtgacgctagcacacgccttggttttttgaaggtgtggacttcaattgctaaatcggggggtgatcaatcacttttaatgaatggatcactcccggttttagatagattgattgacgttccaaaactagatgaatacagattgaggcacacaagccttgaaactttttatcagcctccgagccttgttgggcctacaaagaaagttcgggcaacatttatattgagtcgaaccaatatcaatcagttaaagaaaagggtccttacccaaatcccaacattggaatacatatcatcttttacggtaacttgtggttatatatggagttgcattgcaaaatcactagtgaaaatgggagaaaaaaagggcgaggatgagttagaacaattcatttgcacggctgattgtcgctctcgtatggatccaccgattccatcaacctactttggtaattgtggtgcaccatgtgtcacaaccataaaaaatgttgttttgtcgagtgaaaatggatttgtattcgctgctaaactaattggcgaggctataaataaaatggtaaagaataaggaaggaatcttgaaagatgccgagagatggcatgatgctttcaagattccagcaaggaagattggtgttgcgggtacaccaaagctcaacttctatgatattgattttgggtgggggaagccgcaaaagaatgaaactatttcgattgattataacggttcggttgctataaatgcaagcaaagaatcaacacaagattttgaaattggattgtgtttttcaaatatgcaaatggaggcgtttgctgatatctttaatcacggattagagagtgagatataa

SEQ ID NO: 4 - protein, 459 aa, Chrysanthemum x morifolium
MASNSIVTILEQSRISSPPGTIGERSLPLTFFDIGWVPFPPVHHVFFYRFPHSKSHFLETVVPNLKHSLSLALQHFFPFASNLYVSPNADDFGVIRKPEIRHVEGDYVALTFAECSLDFNDLTGNHPRKCENFYPLVPPLGNVVKMADCVTIPLFSVQVTYFRDSGISIGMTNHHSLGDASTRLGFLKVWTSIAKSGGDQSLLMNGSLPVLDRLIDVPKLDEYRLRHTSLETFYQPPSLVGPTKKVRATFILSRTNINQLKKRVLTQIPTLEYISSFTVTCGYIWSCIAKSLVKMGEKKGEDELEQFICTADCRSRMDPPIPSTYFGNCGAPCVTTIKNVVLSSENGFVFAAKLIGEAINKMVKNKEGILKDAERWHDAFKIPARKIGVAGTPKLNFYDIDFGWGKPQKNETISIDYNGSVAINASKESTQDFEIGLCFSNMQMEAFADIFNHGLESEI

SEQ ID NO: 5 - DNA, 1383 bp, Chrysanthemum x morifolium
atggcttccaatcccattgttacaattcttgaacactctcgaatatcctcaccaccaggcaccattggtgaacgatcattgccgcttacttttttcgacattacatggctagctttccctcctgtccatcatgttttcttctacgccttccagcactctaaatcacatttcctagaaaccgttgttccaaatcttaagcactctttatcactcacactacaacattttttcccattcgcaggcaatttgtttgtatttcctaatgctgatgattatggtgtcattagaaaaccggaaattcgacacgtggaaggtgattatgttgcacttacttttgcagaatgtcctactcttgattttaatgatttgacaggaaaccatcctcgagaatgtgaaaattttcatccacttgtacctccattgggtgatcttgttaaaatgtatgattatgtcacgatcccacttttgtcggtccaagttacgtactttaaggactcgggtatctctattggtatgacaaatcatcataccgtagctgatgctagcacacgccttggttttttgaaggcgtggacttcaattgctaaatcggggagagatcaatcatttgtaatgaatggatcacccccggttttagataggttgattgacattccaaaactagatgaattcagattgaggcacacaagccttgaaactctttatcagcctcggagccttgttgggcctactaagaaagttcgggcaacatttatattgagtcgaaccaatatcaatcagttgaagaaaagggttctcacccaaatcccaacattggaatacatatcatcttttacggtaacttgtggttatatatggagttgcattgcgaaatcactagtgaaaatgggagaaaaaaagggtgaggacgagttagaacaattcattatctcggttgattgtcgctctcgtatgaatccaccgattccatcaacctacttgggtaattgtggtgcaccatgtatcacaaccataaaaaatgttgttttgtcgagtgaaaatggatttgtattcactgctaaactaattagtgaggctataaataaaatggttaagaataaggaaggaatcttgaaagatgccgagagatggcatgagggtttcaagattccagcaaggaagattggtgttgcgggtacaccaaagctcaacttctatgatatcgattttgggtgggggaagccgcaaaagtacgaagccatttcgattgattataagggttcggttgctataaatgcaagcaaagaatcaacacaagattttgaaattggattgtgttttccaaatatgcaaatgaaggcgtttgctgatatctttaatcacggatttgagagtgaagtataa

SEQ ID NO: 6 - protein, 460 aa, Chrysanthemum x morifolium
MASNPIVTILEHSRISSPPGTIGERSLPLTFFDITWLAFPPVHHVFFYAFQHSKSHFLETVVPNLKHSLSLTLQHFFPFAGNLFVFPNADDYGVIRKPEIRHVEGDYVALTFAECPTLDFNDLTGNHPRECENFHPLVPPLGDLVKMYDYVTIPLLSVQVTYFKDSGISIGMTNHHTVADASTRLGFLKAWTSIAKSGRDQSFVMNGSPPVLDRLIDIPKLDEFRLRHTSLETLYQPRSLVGPTKKVRATFILSRTNINQLKKRVLTQIPTLEYISSFTVTCGYIWSCIAKSLVKMGEKKGEDELEQFIISVDCRSRMNPPIPSTYLGNCGAPCITTIKNVVLSSENGFVFTAKLISEAINKMVKNKEGILKDAERWHEGFKIPARKIGVAGTPKLNFYDIDFGWGKPQKYEAISIDYKGSVAINASKESTQDFEIGLCFPNMQMKAFADIFNHGFESEV

SEQ ID NO: 7 - DNA, 21 bp, Artificial Sequence
  Forward primer for Dm3MaT3
atggcttctcttcccatcttg

SEQ ID NO: 8 - DNA, 21 bp, Artificial Sequence
  Reverse primer for Dm3MaT3
ttaaaggtatgcttttagtcc